(19) United States
(12) Patent Application Publication
(10) Pub. No.: US 2006/0104902 A1
(43) Pub. Date: May 18, 2006

(54) Method of preselection patients for anti-VEGF, anti-HIF-1 or anti-thioredoxin therapy

(76) Inventors: Garth Powis, Houston, TX (US); Lynn Kirkpatrick, Houston, TX (US); Robert J. Gillies, Tucson, AZ (US); Benedicte Jordan, Brussels (BE)

Correspondence Address: Pepper Hamilton LLP;One Mellon Center, 50th Floor, 500 Grant Street, Pittsburgh, PA, 15219, US

(21) Appl. No.: 11/206,526

(22) Filed: Aug. 17, 2005

Related U.S. Application Data
(60) Provisional application No. 60/602,151, filed on Aug. 17, 2004.
(60) Provisional application No. 60/602,163, filed on Aug. 17, 2004.

(51) Int. Cl. A61K 49/00; A61K 39/395; A61K 31/4166

(52) U.S. Cl. 424/9.1; 424/145.1; 514/398

(57) ABSTRACT

The present invention generally relates to methods of preselecting patients for treatment with an anti-VEGF therapy, anti-HIF-1 therapy or anti-thioredoxin therapy. Aspects of the invention combine methods of dynamic contrast enhanced-MRI and diffusion weighted-MRI for the detection of tumor histology. The methodology disclosed herein detects tissue blood volume, tumor vascularity, and abnormal capillary permeability, thereby determining tumor vascularity to determine whether a patient should be administered such therapy.

### CROSS REFERENCE TO RELATED APPLICATIONS

This application claims priority under 35 U.S.C. §119 based upon U.S. Provisional Patent Application No. 60/602,151 entitled “Non-invasive dynamic contrast and diffusion weighted magnetic resonance imaging to predict patient response to therapy with anti-HIF-1 therapy and to preselect patients for treatment with anti-HIF-1 and anti-angiogenic therapy” filed Aug. 17, 2004 and U.S. Provisional Patent Application No. 60/602,163 entitled “Monitoring Effects of PX-12 on Tumor Vascular Permeability” filed Aug. 17, 2004.

### GOVERNMENT INTERESTS

The United States Government may have certain rights to this invention pursuant to work funded under PHS grants U54 CA90821, CA077575 and infrastructure grants R24 CA083 148 and P30 CAQ3074, CA98920.

### BACKGROUND

Solid tumors with areas of hypoxia are the most aggressive and difficult tumors to treat). Moreover, the common, slow-growing solid tumors are resistant to most cytotoxic drugs. Among several factors influencing resistance is the degree of intra-tumoral hypoxia. The proportion of hypoxic cells in a tumor is, in part, a function of tumor size, but even small tumors (about 1 mm in diameter) may have hypoxic fractions ranging from about 10-30%. Additionally, micrometastases may have areas of hypoxia at the growing edge where tumor growth outstrips new blood vessel formation. The tumor types in which significant hypoxic fractions have been identified include all the common solid tumors, such as, but not limited to, lung, colon, head and neck and breast cancers.

Hypoxic cancer cells survive the hostile hypoxic environment by changing to a glycolytic metabolism, becoming resistant to programmed cell death (apoptosis), and producing factors such vascular endothelial growth factor (VEGF) that stimulate new blood vessel formation from existing vasculature (angiogenesis), leading to increased tumor oxygenation and growth. The cancer cell response to hypoxia is mediated through the hypoxia inducible factor-1 (HIF-1) transcription factor.

HIF-1 is a heterodimeric molecule composed of a labile alpha (HIF-α) and a constitutive beta (HIF-1β) subunit, both members of the basic-helix-loop-helix Per-ARNT-SIM (PAS) family of transcription factors. One partner, HIF-1β, is the aryl hydrocarbon receptor nuclear translocator (Arnt). HIF-1β is constitutively expressed, it is stable, and its levels are not altered by hypoxia, it is equivalently expressed in normoxia and hypoxia. In contrast, HIF-1α is constitutively expressed, but under aerobic conditions (normoxia, i.e., normal oxygen conditions) it is rapidly degraded by the ubiquitin-26S proteasome pathway so that HIF-1α levels are almost non-detectable. HIF-1α expression, and subsequently HIF-1 transcriptional activity, increases exponentially as cellular oxygen concentration is decreased (hypoxia). Under conditions of hypoxia, HIF-1α degradation is inhibited and HIF-1α protein levels increase resulting in an increase in HIF-1 transactivating activity. Thus the major regulation of the transcriptional activity of HIF-1 is due to the HIF-1α component.

HIF-1α and HIF-1β associate in the cytosol prior to transport to the nucleus where they bind to hypoxic regulated element (HRE) DNA sequences in the 3′ and 5′ regions of hypoxia regulated genes. Several dozen target genes that are transactivated by HIF-1 have been identified, including, but not limited to, erythropoietin, glucose transporters, glycolytic enzymes, as well as genes increasing tissue perfusion such as vascular endothelial growth factor (VEGF), inducible nitric oxide synthase, and erythropoietin.

HIF-1α degradation is mediated by an approximately 200-amino acid domain that has been termed the “oxygen-dependent degradation domain” (ODD). Cells transfected with cDNA encoding HIF-1α in which the ODD is deleted demonstrate constitutively active HIF-1α protein regardless of oxygen tension.

HIF-1α is required for both embryonic development and growth of tumor explants, which underscores a central role of this molecule in the hypoxic response in vivo. In adult animals, HIF-1α is overexpressed in many types of cancers (such as epithelial and high-grade pre-malignant lesions), ischemic tissue (such as muscle, brain, heart, etc), and healing wounds.

HIF-1α expression has been detected in the majority of solid tumors examined including brain, bladder, breast, colon, ovarian, pancreatic, renal and prostate, whereas no expression was detected in surrounding normal tissue, nor was it detected in benign tumors. Clinically, HIF-1α over-expression has been shown to be a marker of highly aggressive disease and has been associated with poor prognosis and treatment failure in a number of cancers including breast, ovarian, cervical, oligodendroglioma, esophageal, and oropharyngeal.

HIF-1α presence correlates with tumor grade as well as vascularity. High-grade glioblastoma multiforme (GBM) tumors have significantly higher levels of VEGF expression and neovascularization compared with low-grade gliomas. Studies such as these suggest that HIF-1 mediates hypoxia-induced VEGF expression in tumors leading to highly aggressive tumor growth.

In addition, the thioredoxin redox couple thioredoxin/thioredoxin reductase (TR/Trx) is a ubiquitous redox system found in both prokaryotic and eukaryotic cells. The thioredoxin system is comprised primarily of two elements: thioredoxin and thioredoxin reductase. Thioredoxins are a class of low molecular weight redox proteins characterized by a highly conserved Cys-Gly-Pro-Cys-Lys active site. The cysteine residues at the active site of thioredoxin undergo reversible oxidation-reduction catalyzed by thioredoxin reductase. Trx-1 is ubiquitously expressed with a conserved catalytic site that undergoes reversible NADPH-dependent reduction by selenocysteine-containing flavoprotein Trx-1 reductases.

The redox protein thioredoxin-1 (Trx-1) has been proven to be a rational target for anticancer therapy involved in promoting both proliferation and angiogenesis, inhibiting apoptosis, and conferring chemotherapeutic drug resistance. Trx-1 was originally studied for its ability to act as a reducing cofactor for ribonucleotide reductase, the first unique step in DNA synthesis. Thioredoxin also exerts specific redox control over a number of transcription factors to modulate their DNA binding and, thus, to regulate gene transcription. Transcription factors regulated by thioredoxin include, but are not limited to, NF-kB, p53, TFIIIC, BZLF1, the glucocorticoid receptor, and hypoxia inducible factor 1α (HIF-1α). Trx-1 also binds in a redox-dependent manner and regulates the activity of enzymes such as apoptosis signal-regulating kinase-1 protein kinases C δ, \acute{ε}, ξ, and the tumor suppressor phosphatase PTEN.

Trx-1 expression is increased in several human primary cancers, including, but not limited to, lung, colon, cervix, liver, pancreatic, colorectal, and squamous cell cancer. Clinically increased Trx-1 levels have been linked to aggressive tumor growth, inhibition of apoptosis, and decreased patient survival. The importance of redox regulation of transcription factor activity can be illustrated by its effect on HIF-1α expression. Trx-1 overexpression has been shown to increase HIF-1α protein levels and to increase HIF-1 transactivating activity under both normoxic and hypoxic conditions.

### SUMMARY OF THE INVENTION

Angiogenesis is the growth of new blood vessels. This process is normally under tight regulation. In cancer, more particularly malignant tumors, the abnormal growth also induces the abnormal stimulation of new blood vessels. This can be detected by measuring plasma or tumor levels of biomarkers that may be altered. Alternatively, MRI technologies may be used to monitor vascular permeability, vascular volume, and cell volume fraction.

Embodiments of the invention provide methods of using DCE-MRI and DW-MRI for determining tumor vascular structure to determine whether an individual should be treated with anti-VEGF therapy, anti-HIF-1 therapy or anti-thioredoxin therapy or a combination thereof.

Further embodiments provide methods of determining the effects of anti-VEGF therapy, anti-HIF-1 therapy or anti-thioredoxin therapy on tumor vasculature. In one embodiment, the method comprises administering large molecular weight contrast agents and measuring tumor blood flow. The change in tumor blood flow correlate with changes in tumor vascularity, and thus the efficacy of the anti-VEGF therapy, anti-HIF-1 therapy or anti-thioredoxin therapy.

In another embodiment, the method comprises measuring the movement of water molecules following administration of anti-VEGF therapy, anti-HIF-1 therapy or anti-thioredoxin therapy. This allows for the measurement of cellular volume and any changes in cellular volume that may have occurred due to the effect of the therapy on the tumor. Further embodiments combine both the DCE-MRI and DW-MRI methods to analyze the tissue blood volume, tumor vascularity, and capillary permeability to determine changes in tumor vascular structure due to the anti-VEGF therapy, anti-HIF-1 therapy or anti-thioredoxin therapy.

Embodiments of the invention wherein patients are screened and preselected for a therapy are also described. Although tumors may be of the same histopathologic type, their susceptibility to a therapeutic compound and/or therapeutic regimen may differ. Thus, embodiments wherein a tumors sensitivity to a therapeutic compound, preferably anti-VEGF therapy, anti-HIF-1 therapy or anti-thioredoxin therapy, more preferably anti-VEGF agents such as antibodies and small molecules, anti-thioredoxin agents and anti-HIF agents, are determined using DCE-MRI and DW-MRI to screen the effects the therapeutic compound and/or therapeutic regimen on tumor vascular structure.

### DESCRIPTION OF THE DRAWINGS

The file of this patent contains at least one photograph or drawing executed in color. Copies of this patent with color drawing(s) or photograph(s) will be provided by the Patent and Trademark Office upon request and payment of necessary fee.

In part, other aspects, features, benefits and advantages of the embodiments of the present invention will be apparent with regard to the following description, appended claims and accompanying drawings where:

FIG. 1. DW images at a b value of 25 (up) and corresponding diffusion maps (bottom) of a HT-29 tumor bearing mouse before, 24 h, and 48 hours after PX-478 injection. Each image represents an axial slice of the mouse with the tumor area encircled and indicated by an arrow.

FIG. 2. Summed ADCw histograms of control (filled bars), and treated tumors (open bars) at each timepoint. A right shift in tumor ADCw is observed at 24 and 48 h post-treatment.

FIG. 3. Full time course of average tumor ADCw following PX-478 administration (control mice, full line; and treated mice, dotted line). A significant increase in average tumor ADCw is observed at 24 and 36 h post-treatment.

FIG. 4A. Permeability maps of tumors 2, 12, 24, and 48 hours after either vehicle (control) or drug (PX-478) injection. Each image represents an axial slice of the mouse with the tumor area encircled. A substantial reduction in tumor vascular permeability is observed as soon as 2 hours after PX-478 injection and until 24 h, in comparison with the control situation. This is no longer observed by 48 hours after treatment. 4B. Vascular volume fraction (VV) maps of tumors 2, 12, 24, and 48 hours after either vehicle (control) or drug (PX-478) injection. Each image represents an axial slice of the mouse with the tumor area encircled. Some individual positive or negative changes can be observed but these were not significant between groups.

FIG. 5. Full time course of average vascular permeability (A) and vascular volume fraction (B) following administration of PX-478 (control mice, full line; and treated mice, dotted line). Blood vessel permeability was estimated from the slope of the enhancement curves, and tumor vascular volume (VV) fraction was estimated from the ordinate. A significant reduction in permeability is observed 2, 12, and 24 h after treatment with PX-478, while no changes are observed in the VV fraction.

FIG. 6. Summed permeability histograms of control (filled bars, n=4) and treated tumors (open bars, n=4) at each time point. Note that the median (dotted line) of treated tumors is lower than the median value of the controls. It is progressively shifted to the median of the controls over time, and is back at control values 48 h post-treatment.

FIG. 7A. Relative change in HT-29 tumor vascular permeability and vascular volume fraction one hour after treatment with anti-VEGF antibody (Avastin™ (bevacizumab)). A significant reduction in permeability as well as in VV fraction is observed with this positive control. 7B. Relative change in A-549 tumor (resistant to the antitumor activity of PX-478, negative control) vascular permeability and vascular volume fraction two hours after treatment with PX-478. No significant change is observed in the DCE parameters.

FIG. 8. HIF-1α levels and antitumor activity of PX-478 in HT-29 human colon cancer and A-549 non small cell lung cancer xenografts in scid mice. Male scid mice were injected sc with A, 10⁷ HT-29 human colon cancer cells or B, A-549 non small cell lung cancer cells. The HT-29 tumors were allowed to grow to 400 mm³ and the A-549 tumors to 360 mm³ and treatment begun with (O) vehicle alone or (□) PX-478 at 80 mg/kg ip daily for 5 days for HT-29 xenografts and 100 mg/kg ip daily for 5 days for A-549 xenografts. The upper panels show typical immunohistochemical staining for HIF-1α in the untreated tumor xenografts at the start of the study. The lower panels show tumor xenograft growth curves. There were 8 mice in each group and bars are SE.

FIG. 9A. Permeability maps of tumors 2 hours after vehicle (control) or PX-12 injection (Tx). Each image represents an axial slice of the mouse with the tumor area encircled and indicated by an arrow. Note the substantial reduction in tumor vascular permeability in treated tumors (bottom) in comparison with control tumors (top). FIG. 9B. Vascular Volume (VV) fraction maps of tumors 2 hours after vehicle (control) or PX-12 injection (Tx). Each image represents an axial slice of the mouse with the tumor area encircled and indicated by an arrow. No obvious change in the average VV fraction after treatment is visible.

FIG. 10. Summed histograms of control (open bars, 3mice/group), and treated tumors (open bars, 3 mice/group) at each timepoint. Note that the median (dotted line) of treated tumors is lower than the median value of the controls. It is progressively shifted to the median of the controls over time, and is back at control values 48 h post-treatment.

FIG. 11. Full time course of average tumor vascular permeability (A) and VV fraction (B) following PX-12 administration (3 control mice, dotted line; and 3 treated mice, full line). Blood vessel permeability was estimated from the slope of the enhancement curves, and tumor vascular volume (VV) fraction was estimated from the ordinate. A significant reduction in permeability is observed 2, 12, and 24 h after treatment with PX-12, while no changes are observed in the VV fraction.

FIG. 12. Human VEGF levels (pg/μg protein) in HT-29 xenografts, and VEGF levels (pg/ml) in plasma after treatment with PX-12, 25 mg/kg ip (n=4 mice per time point). A significant decrease in mouse VEGF in plasma (P<0.02) and human VEGF in tumors (P<0.001) was observed after 24 h of PX-12 treatment.

### DESCRIPTION OF THE INVENTION

Before the present compositions and methods are described, it is to be understood that this invention is not limited to the particular molecules, compositions, methodologies or protocols described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference. Nothing herein is to be construed as an admission that the invention is not entitled to antedate such disclosure by virtue of prior invention.

It must also be noted that as used herein and in the appended claims, the singular forms “a”, “an”, and “the” include plural reference unless the context clearly dictates otherwise. Thus, for example, reference to a “cell” is a reference to one or more cells and equivalents thereof known to those skilled in the art, and so forth.

As used herein, the term “about” means plus or minus 10% of the numerical value of the number with which it is being used. Therefore, about 50% means in the range of 45%-55.

“Contrast media” refers to compounds that can be used to resolve adjacent tissues which are similar when imaging but histologically or physiologically different.

“Imaging” refers to a method of examining tissue by exposing the tissue to energetic waves and measuring the differences in absorption of the energy transmitted or by measuring the release of energy by the tissues in the presence of the energetic waves.

“Interstitial space of a tumor” refers to the area between cells in a solid tumor exclusive of vascular spaces.

“Optional” or “optionally” means that the subsequently described event or circumstance may or may not occur, and that the description includes instances where the event occurs and instances where it does not.

As used herein, the terms “pharmaceutically acceptable”, “physiologically tolerable” and grammatical variations thereof, as they refer to compositions, carriers, diluents and reagents, are used interchangeably and represent that the materials are capable of administration upon a mammal without the production of undesirable physiological effects such as nausea, dizziness, rash, or gastric upset. In a preferred embodiment, the therapeutic composition is not immunogenic when administered to a human patient for therapeutic purposes.

“Providing” when used in conjunction with a therapeutic or diagnostic means to administer a therapeutic directly into or onto a target tissue or to administer a therapeutic or diagnostic to a patient whereby the therapeutic or diagnostic positively impacts the tissue to which it is targeted.

As used herein “subject” or “patient” refers to an animal or mammal including, but not limited to, human, dog, cat, horse, cow, pig, sheep, goat, chicken, monkey, rabbit, rat, mouse, etc.

As used herein, the term “therapeutic” means an agent utilized to treat, combat, ameliorate, prevent or improve an unwanted condition or disease of a patient. The methods herein for use contemplate prophylactic use as well as curative use in therapy of an existing condition.

The terms “therapeutically effective” or “effective”, as used herein, may be used interchangeably and refer to an amount of a therapeutic composition embodiments of the present invention. For example, a therapeutically effective amount of a composition comprising anti-VEGF therapy is a predetermined amount calculated to achieve the desired effect, i.e., to effectively inhibit VEGF expression in an individual to whom the composition is administered.

The term “unit dose” when used in reference to a therapeutic composition of the present invention refers to physically discrete units suitable as unitary dosage for the subject, each unit containing a predetermined quantity of active material calculated to produce the desired therapeutic effect in association with the required diluent; i.e., excipient, carrier, or vehicle.

Cancers are diseases that cause cells in the body to change and grow out of control. One feature that is prevalent in malignant tumors is angiogenesis, where the cancer mimics the body's ability to generate new vasculature to supply blood to the tumor. The tumor vasculature is different from normal vascular tissue in that capillaries in tumor regions tend to be more porous than normal capillaries.

One embodiment of the present invention provides methods of screening and preselecting patients for anti-VEGF therapy, anti-HIF-1 therapy or anti-thioredoxin therapy by administering a macromolecular contrast medium to the patient; imaging the change in signal intensity of diffusion weighted and spin-echo weighted images over time in a tumor to obtain a signal intensity; and determining changes in tumor vascular structure. If the tumor vascular structure is permeable, the patient may be entered into a therapeutic regimen. The therapeutic regimen may comprise administering a therapeutically effective amount of an anti-VEGF therapy, anti-HIF-1 therapy or anti-thioredoxin therapy or agent. Exemplary agents include anti-VEGF antibodies, thioredoxin inhibitors and HIF inhibitors.

In one embodiment, a preferred thioredoxin inhibitor is a an asymmetric disulfide, more preferably 1-methylpropyl 2-imidazolyl disulfide, herein designated as PX-12, with the general formula of:

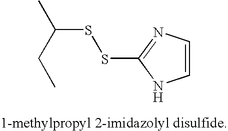

In another embodiment, a preferred HIF inhibitor is S-2-amino-3-[4′-N,N,-bis(2-chloroethyl)amino]-phenyl propionic acid N-oxide dihydrochloride, herein designated PX-478, with the general formula of:

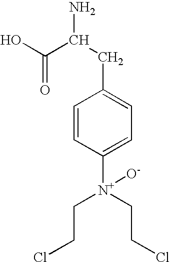

Another embodiment provides methods for the early detection and analysis of tumor vascularity by determining the vascular permeability, vascular volume and cell volume fraction. Various embodiments provide methods of magnetic resonance imaging (MRI) and combinations of MRI technologies for the detection of tumor vascularity.

In order for chemotherapy to be effective, the medications should destroy tumor cells and spare the normal body cells which may be adjacent. This is accomplished by using medications that affect cell activities that go on predominant in cancer cells but not in normal cells. One difference between normal and tumorous cells is the amount of oxygen in the cells. Many tumorous cells are oxygen deficient and are “hypoxic”.

Another difference between normal and tumorous tissue is related to this lack of oxygen. Reductive metabolic processes may be more prevalent in the hypoxic environment of solid tumors. Reductive enzymes reduce functional groups (such as N-oxides) having a potential to be reduced. Nitro compounds are reduced to amino derivatives and quinones are reduced to hydroquinones by enzymes such as DT-diaphorase, cytochrome P₄₅₀, cytochrome P₄₅₀ reductase and xanthine oxidase.

These two differences between normal and tumorous cells has led to the development of bioreductive antitumor drugs. These are drugs which exploit the hypoxic nature, and the reductive nature, of tumorous cells. These drugs are nontoxic and inactive until they are reduced by hypoxic cells thereby becoming toxic and active, cytotoxic agents.

A number of N-oxides have been examined recently for this bioreductive activity. One is the N-oxide derivative of 1,4-bis-{[2-(dimethyl-amino)ethyl]amino} 5,8-dihyroxyanthracene-9,10-dione. This N-oxide is more toxic in vivo under conditions that promote transient hypoxia or which diminish the oxic tumor fraction. Others are the mono-N-oxides of fused pyrazines. The N-oxide function is essential for the differential cytotoxic properties of these agents. Another is the aliphatic N-oxide of nitacrine. It has an exceptionally high selectivity for hypoxic cells (approximately 1500 fold) and an improved ability to diffuse into the extravascular compartment of tumors. The N-oxide of these agents itself does not provide a reactive species but the reduction of this functional group unmasks an agent with cytotoxic potential. However, so far, none of these N-oxides has been found to have clinical activity and to lack toxicity to normal cells and tissue.

One N-oxide derivative which has been studied as an anti-tumor agent is the N-oxide derivative of chlorambucil (also known as a nitrogen mustard derivative). Chlorambucil is toxic to tumorous cells. Chlorambucil acts as an anti-tumor agent by cross-linking (or alkylating) DNA, thus preventing DNA from replicating and cells from growing. Chlorambucil has this effect in both tumorous and normal cells (i.e., those that are actively dividing).

Alkylating agents as a group have had problems with side effects. Because chlorambucil is relatively slow acting, fewer side effects have been an issue with this medication.

N-oxide derivatives of chlorambucil are less cytotoxic than chlorambucil and under hypoxic conditions the cytotoxicity is potentiated by the presence of hypoxia proteins such as HIF-1α. N-oxide derivatives of chlorambucil which are stable in hypoxic and oxic cells, are toxic in cells having varying degrees of hypoxia, and show little toxicity to oxic cells, have been and are being developed, some of which are described in U.S. Pat. No. 5,602,278, which is incorporated by reference in its entirety.

An example of another N-oxide nitrogen mustard derivative is S-2-amino-3-[4′N,N,-bis(2-chloroethyl)amino]-phenyl propionic acid N-oxide dihydrochloride, herein designated PX-478. This compound is a novel agent that suppresses both constitutive and hypoxia-induced levels of HIF-1α in cancer cells. The inhibition of tumor growth by PX-478 is positively associated with HIF-1α levels in a variety of different human tumor xenografts in scid mice. Inhibition of HIF-1α is associated with reduced hypoxic induction of a HIF-1 target gene VEGF, a key angiogenic factor. Inhibition of VEGF expression results in the reduction or loss of tumor angiogenesis, with the resultant changes in tumor vascular structure. Thus, embodiments of the invention provide methods that allow early detection of changes in tumor vascular structure, thereby allowing for the determination of the efficacy of a therapeutic compound and/or therapeutic regimen.

One embodiment of the present invention relates to asymmetric disulfides. More specifically, an aspect of the present invention relates to compounds or mixtures of compounds which include an asymmetric disulfide or biological equivalent thereof which interacts, interferes, inhibits, or competes with redox systems, particularly redox systems involving proteins having cysteine residues, and more particularly to redox systems involving thioredoxin and/or thioredoxin reductase.

As used herein, the term asymmetric disulfide means any compound having a sulfur-sulfur linkage which is not a mirror image of itself when split down the sulfur-sulfur. When speaking of a particular asymmetric disulfide, the term includes all biochemical equivalents (i.e. salts, precursors, and basic form) of the particular asymmetric disulfide being referenced (i.e., reference to n-butyl imadazolyl disulfide includes the salt thereof). This term specifically includes disulfides having the general formula of R₁ —S—S—R₂ as well as (bis)disulfides having the general formula of R₁ —S—S—Y—S—S—R₂ wherein R₁, R₂, and Y may be any chemical substituent, but is preferably selected from the group consisting of imidazoles, thiadiazolyls, thiazolyls, benzimidazolyls, purinyls, phenyl, benzyl, phenylethyl, pyridine, pyrimidine, benzoxazole, benzthiazolyls, alkyl, cycloalkyl, hydroxylalkyl, carboxyalkyl, haloalkyl, and cycloalkanone.

When the term asymmetric disulfide is used it means that the groups on either side of a disulfide linkage are not the same. In the case of disulfides having the formula R—S—S—R this asymmetric relation may be represented by R₁ —S—S—R₂. In the case of (bis)disulfide compounds although R₁ and R₂ may not be different, and the overall compound may be “symmetrical” around the center of the formula, that is, in the formula R₁—S—S—Y—S—S—R₂, R₁ and R₂ may be the same group, the term asymmetrical as used herein refers to the fact that when either sulfur-sulfur linkage is split down the middle, the disulfides are asymmetrical (i.e. R—S—S—Y—S—}) are not equivalent. By this definition and as used herein all (bis)disulfide compositions are asymmetrical.

The preferred asymmetric disulfides of the present invention include, but are not limited to, imidazolyl disulfide, thiadiazolyl disulfide, mercaptothiadiazolyl disulfide, thiazolyl disulfide, phenyl disulfide, benzyl disulfide, phenylethyl disulfide, nicotinic acid disulfide, pyrimidine disulfide, benzoxazolyl disulfide, benzothiazolyl disulfide, benzimidazolyl disulfide, purinyl disulfide, cycloalkyl disulfide, captopril disulfide, and menthone disulfide.

The asymmetrical disulfides of the present invention have respective R groups of divergent functionality. Preferably in the general formula R₁ —S—S—R₂ one of R₁ or R₂ is a good leaving group and the respective other is a poor leaving group. Examples of good leaving groups are compounds which contain electron withdrawing groups or groups which delocalize the electrons of the functional groups (i.e., aromatic and imidazlyl groups). It is preferable that the aromatic groups of the present invention include heteroatoms such as oxygen, nitrogen, and sulfur. Poor leaving groups do not generally have such electron withdrawing characteristics or delocalized electrons. Thus, they do not form substantially stable species when or if they are cleared from the molecule. An example of a poor leaving group is an unsubstituted alkane or alkyl group. The asymmetrical disulfides of the present invention are particularly useful to treat cancers, more particularly, cancers such as myeloma, cervical, lung, gastric, colon, renal, prostate, and breast cancers.

Several 2-imidazolyl disulfides have been shown to inhibit Trx-I. For example, but not limited to, benzyl 2-imidazolyl disulfide and 1-methylpropyl 2-imidazolyl disulfide, previously described in U.S. Pat. No. 6,552,060, herein incorporated by reference in its entirety. These imidazolyl disulfides are asymmetric disulfides. Among them, 1-methylpropyl 2-imidazolyl disulfide, herein referred to as PX-12, has been identified as a potent inhibitor of the thioredoxin system by irreversibly thioalkylating a critical cysteine residue (Cys⁷³) that lies outside the conserved redox catalytic site of Trx-1. PX-12 is active as a Trx-1 inhibitor at submicromolar concentrations and has been shown to have in vivo antitumor activity against human tumor xenografts in SCID mice. More recently, PX-12 has been shown to cause significant decreases in the expression of HIF 1α and VEGF and microvessel density in xenograft tumors.

Magnetic resonance imaging (MRI) is a noninvasive technique that can be used to obtain information regarding tumor vascularization, metabolism, and pathophysiology, and allows early assessment of the therapeutic effects of cancer drugs. One method in which to study tumor angiogenesis is dynamic contrast enhanced magnetic resonance imaging (DCE-MRI) which measures tumor vascular characteristics after administration of a contrast medium. DCE-MRI is the acquisition of sequential images during the passage of contrast agent within a tissue of interest. DCE-MRI is an effective method in the early detection and classification of a cancer by analyzing the vascular structure.

DCE-MRI is a computer-enhanced modality that relies on a special algorithm to estimate blood flow. The ability to measure blood flow allows for the ability to see changes in tumor vascularity, which occur at a much earlier stage in the treatment of tumors than does shrinkage of tumor mass, as measured with a caliper for example. Using DCE-MRI to estimate drug efficacy represents an improvement over traditional marker analyses of tumor biopsy specimens, which are not only invasive but also subject to sampling bias. Currently, oncologists measure vascular growth by analyzing the markers of angiogenesis, such as circulating levels of the proangiogenic molecules such as basic fibroblast growth factor (FGF) and vascular endothelial growth factor (VEGF). The levels of proangiogenic molecules may not provide accurate prediction of the response of certain patients to a particular therapeutic agent. Embodiments of the invention use DCE-MRI to provide methods for the earlier assessment of the response of a particular tumor to a therapeutic compound and/or therapeutic regimen.

MRI enhanced with small molecular weight contrast agents is extensively used in the clinic to differentiate benign from malignant lesions as well as to monitor tumor microvascular characteristics during treatment. However, the advantage of using large molecular agents (macromolecular contrast media, MMCM) designed for prolonged intravascular retention has been demonstrated in several preclinical studies. MMCM show a leak into the interstitium of carcinomas, whereas they are confined to the intravascular space in benign tumors, thereby allowing for the classification of a tumor. Correlations between MMCM enhanced parameters and angiogenic markers such as microvessel density and VEGF levels have previously been studied.

Although “macromolecular MRI contrast media” (MMCM) have been known to those of skill for some time, these media have only recently found diagnostic uses. These media typically contain chelated gadolinium groups conjugated to proteins, such as albumin. These types of contrast agents, because they do not cross healthy blood vessel walls, have allowed investigators to gauge the endothelial permeability of tumor vessels compared to the permeability of vessels in healthy tissues.

Of interest are contrast agents used for imaging the blood pool and monitoring its movement. MRI imaging assisted by such agents is useful for such procedures as assessments of relative tissue blood volume, estimation of tumor vascularity or tissue perfusion, and detection of abnormal capillary permeability. Clinical applications include assessment of neoplasia. The contrast agents should remain in the blood vessels and capillaries rather than leaving it through such means as diffusion into extravascular compartments. Aspects of the invention utilize contrast agents of a relatively high molecular weight, generally on the order of greater than about 20 kD, in other embodiments the molecular weight may be about 30 kD or more, which prevents the agents from diffusing through capillaries. Other embodiments may contain contrast media of smaller molecular weights yet retaining the effective molecular sizes of about 30 kD. This can be effected by the binding of smaller contrast media, after injection, to larger molecules within the body, particularly albumin. A further advantage of MMCM is that the prolonged intravascular retention of these agents permits imaging of the blood pool in multiple body regions without repeated dosing.

Several classes of compounds have been explored as potential contrast agents. For MRI, these classes include superparamagnetic iron oxide particles, nitroxides, and paramagetic metal chelates (such as gallidium). See, Mann J. S. and Brasch R. C. in HANDBOOK OF METAL-LIGAND INTERACTIONS IN BIOLOGICAL FLUIDS: BIORGANIC MEDICINE VOL. 2, Berthon, G., ed., Marcel Dekker, Inc., New York, N.Y. (1995).

The MCMM may also include contrast agents attached to a large backbone. The backbone can be a protein, such as albumin, a polypeptide, such as poly-L-lysine, a polysaccharide, a dendramer, or a rigid hydrocarbon or other compound with a small molecular weight but a larger effective molecular size. The preferred backbones of this invention are compounds that when passed through a gel filtration matrix, behave similarly to a peptide of about 20 kD to about 30 kD.

MMCM that is formed in vivo is also included. A contrast medium may be administered to a subject and the medium attaches to a large backbone, such as albumin or polysaccharides.

Because the capillary endothelium of tumors and injured tissues exhibit high permeability rates relative to normal tissue, MMCM passively diffuses into these tissues. The poorly developed or absent lymphatic system of tumors and some tissues limits the rate of movement of macromolecules out of these tissues. This combination (enhanced permeability and retention) is used during imaging of these tissues. The tumors and injured tissues are seen by imaging as a time-dependent increased intensity in the interstitial space. The prolonged retention within the vascular compartment of tumors and some injured tissues provides nearly a constant level of enhancement.

In MRI, contrast media improve the image obtained by altering hydrogen protons. In the presence of an external magnetic field, protons produce a weak fluctuating field which is capable of relaxing neighboring protons. This situation is dramatically altered in the presence of a strong paramagnet (such as a contrast agent). A single unpaired electron in a contrast agent induces a field which is nearly 700 times larger than that produced by protons and fluctuates with a frequency component which is in a range that profoundly affects nearby protons. Thus in a weighted imaging sequence, the paramagnetic contrast media causes the protons of nearby hydrogen nuclei to release far greater amounts of energy to reach equilibrium after a radio frequency pulse and appear as very bright areas in an MRI image. The protons in tissues that take up the contrast medium release less energy to reach equilibrium and appear darker in an MRI.

Normally, paramagnetic lanthanides and transition metal ions are toxic in vivo. Therefore, it is necessary to incorporate these compounds into chelates with organic ligands. Acceptable chelates include: 1,4,7,10-tetraazacyclododecane-N,N′,N″,N′″-tetraacetic acid (DOTA); 1,4,7,10-tetraazacyclododecane-N,N′,N″-triacetic acid (DO3A), 1,4,7-tris(carboxymethyl)-10-(2-hydroxypropyl)-1,4,7,10-tetraazacyclododecane (HP-DO3A), and more preferably, diethylenetriaminepentaacetic acid (DPTA).

Paramagnetic metals of a wide range are suitable for chelation. Suitable metals are those having atomic numbers of 22-29 (inclusive), 42, 44 and 58-70 (inclusive), and having oxidation states of 2 or 3. Those having atomic numbers of 22-29 (inclusive), and 58-70 (inclusive) are preferred, and those having atomic numbers of 24-29 (inclusive) and 64-68 (inclusive) are more preferred. Examples of such metals are chromium (III), manganese (II), iron (II), cobalt (II), nickel (II), copper (II), praseodymium (III), neodymium (III), samarium (III), gadolinium (III), terbium (III), dysprosium (III), holmium (III), erbium (III) and ytterbium (III). Chromium (III), manganese (II), iron (III) and gadolinium (III) are particularly preferred, with gadolinium (III) the most preferred. Gadolinium (Gd) is a lanthanide metal with an atomic weight of 157.25 and an atomic number of 64. It has the highest thermal neutron capture cross-section of any known element and is unique for its high magnetic moment (7.98 at 298° K.). This is reflected in its seven unpaired electrons (CRC HANDBOOK OF CHEMISTRY AND PHYSICS, 75TH ED., Lide, D. R., ed., 1995).

A preferred MMCM is albumin-(Gd-DPTA)₃₀. The molecular weight of albumin-(Gd-DPTA)₃₀ is 92 kD. The distribution volume of albumin-(Gd-DPTA)₃₀ is 0.05 l/kg which closely approximates the blood volume. Plasma half life is approximately 3 hours with a delayed renal elimination over days.

Typically, the administration of contrast media for imaging tumors is parenteral, e.g., intravenously, intraperitoneally, subcutaneously, intradermally, or intramuscularly. Thus, the invention provides compositions for parenteral administration which comprise a solution of contrast media dissolved or suspended in an acceptable carrier, preferably an aqueous carrier. The concentrations of MMCM varies depending on the strength of the contrast agent but typically varies from 0.1 μmol/kg to 100 μmol/kg. A variety of aqueous carriers may be used, e.g., water, buffered water, 0.9% saline, 0.3% glycine, hyaluronic acid and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

Diffusion-weighted MRI (DW-MRI) imaging has steadily evolved from a basic research tool to a clinical tool. Diffusion is a physical property of molecules referring to their ability to move randomly in relation to their thermal energy. Molecular motion is referred to as Brownian motion and it is a random translational movement that occurs at the microscopic level. It is measured in terms of the diffusion coefficient which, in general, increases in more dilute solutions and has a directional component. Since diffusion is a reflection of very small-scale motion, diffusion imaging is very sensitive to motion. Hardware and technical advances have enabled the detection of this very small-scale motion. It represents a major advance in the evolution of pulse sequences that can make subtle abnormalities more obvious and can provide different characterization of tissues and their pathologic processes.

DW-MRI allows noninvasive characterization of biological tissues based on the random microscopic motion of water protons measurement, referred to as the apparent diffusion coefficient of water (ADCw). Preclinical studies have shown that DWI allows early detection of tumor response to chemotherapy. Most likely changes in the diffusion characteristics are caused by a shift of water to the intracellular space. It is therefore anticipated that DW-MRI will detect early changes in cellular volume fractions resulting from apoptosis-associated cell shrinkage, necrosis, or vasogenic edema. Because water is not as diffusionally restricted in the extracellular space, compared to the intracellular, a decrease in cell volume fraction will result in an overall increase in the ADCw. Characterization of the capability of DWI to detect early changes in tumor ADCw following antitumor therapy in preclinical models and in the clinical setting has been previously performed.

A major challenge in tumor biology is to better define the specific characteristics of individual tumors. Tumors sharing a particular histopathologic type may have widely divergent biological properties, such as molecular expression, angiogenesis status, and susceptibility to a therapeutic compound and/or therapeutic regimen. Embodiments of the invention provide methods for the analysis of a patient's tumor using DCE-MRI and/or DW-MRI to define both functional and structural characteristics and responsiveness to a therapeutic compound and/or therapeutic regimen.

Embodiments of the invention are generally directed to methods of quantitatively assessing tumor microvessels using DCE-MRI and/or DW-MRI to non-invasively assay the relative blood volume, microvascular endothelial leakiness, or the interstitial volume of any solid tumor. Thus, aspects of the present invention detect a tumor's malignancy, its angiogenic status, its pathologic grade, and/or the tumors responsiveness to a therapeutic compound and/or therapeutic regimen. Embodiments use MRI and MMCM to screen and preselect patients for anti-VEGF therapy, and further rto detect tumor responses to treatment after initiating therapy.

Further embodiments of the present invention monitor the antitumor activity of PX-478 on HT-29 human colon xenografts using both DCE-MRI and DW-MRI to assess the use of these techniques as early and surrogate endpoints for the antitumor response to the drug. These non-invasive magnetic resonance techniques provide insight on tumor microvessel characteristics, such as permeability and vascular volume fraction, and on cellular volume ratios (cellularity, necrotic fraction), which may be early markers and even predictors of tumor response to a therapeutic agent and/or therapeutic regimen.

The compounds of the invention may be administered in an effective amount to a subject in need of such treatment. As such, the compounds described herein may be useful for the treatment of cancer and other proliferative disorders. Administration of the compounds, in the form of a therapeutic agent, may be carried out using oral, enteral, parenteral or topical administration, including, for example, intravenous, oral, transdermal or any other mode of administration with appropriate vehicle.

Pharmaceutical compositions can be used in the preparation of individual dosage forms. Consequently, pharmaceutical compositions and dosage forms of the invention may comprise the active ingredients disclosed herein (i.e., N-oxide derivatives, preferably derivatives of nitrogen mustards and more preferably S-2-amino-3-[4′-N,N,-bis(2-chloroethyl)amino]-phenyl propionic acid N-oxide dihydrochloride). Further embodiments of the invention may comprise any therapeutic compound and/or therapeutic regiment which is to be assessed for its efficacy in inhibiting a tumor. Pharmaceutical compositions and dosage forms of the invention can further comprise one or more excipients.

Single unit dosage forms of the invention are suitable for oral, mucosal (e.g., nasal, sublingual, vaginal, buccal, or rectal), parenteral (e.g., subcutaneous, intravenous, bolus injection, intramuscular, or intraarterial), or transdermal administration to a patient. Examples of dosage forms include, but are not limited to: tablets; caplets; capsules, such as soft elastic gelatin capsules; cachets; troches; lozenges; dispersions; suppositories; ointments; cataplasms (poultices); pastes; powders; dressings; creams; plasters; solutions; patches; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions, or a water-in-oil liquid emulsions), solutions, and elixirs; liquid dosage forms suitable for parenteral administration to a patient; and sterile solids (e.g., crystalline or amorphous solids) that can be reconstituted to provide liquid dosage forms suitable for parenteral administration to a patient.

The composition, shape, and type of dosage forms of the invention will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990).

Typical pharmaceutical compositions and dosage forms comprise one or more excipients. Suitable excipients are well known to those skilled in the art of pharmacy, and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form. For example, the decomposition of some active ingredients may be accelerated by some excipients such as lactose, or when exposed to water. Active ingredients that comprise primary or secondary amines are particularly susceptible to such accelerated decomposition.

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers, or salt buffers.

Like the amounts and types of excipients, the amounts and specific types of active ingredients in a dosage form may differ depending on factors such as, but not limited to, the route by which it is to be administered to patients. However, typical dosage forms of the invention comprise an amount of from about 1 μg to about 2000 mg, more preferably from about 1 mg to about 1000 mg, even more preferably from about 5 mg to about 500 mg, and more preferably from about 10 mg to about 200 mg.

The compounds of the invention are preferably administered in effective amounts. An effective amount is that amount of a preparation that alone, or together with further doses, produces the desired response. This may involve only slowing the progression of the disease temporarily, although preferably, it involves halting the progression of the disease permanently or delaying the onset of or preventing the disease or condition from occurring. This can be monitored by routine methods. Generally, doses of active compounds would be from about 0.01 mg/kg per day to 1000 mg/kg per day. It is expected that doses ranging from 5-500 mg/kg will be suitable, preferably intravenously, intramuscularly, or intradermally, and in one or several administrations per day.

In general, routine experimentation in clinical trials will determine specific ranges for optimal therapeutic effect for each therapeutic agent and each administrative protocol, and administration to specific patients will be adjusted to within effective and safe ranges depending on the patient condition and responsiveness to initial administrations. However, the ultimate administration protocol will be regulated according to the judgment of the attending clinician considering such factors as age, condition and size of the patient, the compound potencies, the duration of the treatment and the severity of the disease being treated. For example, a dosage regimen of the S-2-amino-3-[4′N,N,-bis(2-chloroethyl)amino]-phenyl propionic acid N-oxide dihydrochloride can be oral administration of from 1 mg/kg to 2000 mg/kg/day, preferably 1 to 1000 mg/kg/day, more preferably 50 to 600 mg/kg/day, in two to four (preferably two) divided doses, to reduce tumor growth. Intermittent therapy (e.g., one week out of three weeks or three out of four weeks) may also be used.

In the event that a response in a subject is insufficient at the initial doses applied, higher doses (or effectively higher doses by a different, more localized delivery route) may be employed to the extent that the patient tolerance permits. Multiple doses per day are contemplated to achieve appropriate systemic levels of compounds. Generally, a maximum dose is used, that is, the highest safe dose according to sound medical judgment. Those of ordinary skill in the art will understand, however, that a patient may insist upon a lower dose or tolerable dose for medical reasons, psychological reasons or for virtually any other reason.

The following methods are used to illustrate the various embodiments of the present invention. The methods are exemplary methods and are not meant to limit the invention.

### EXAMPLE 1

Cell line and tumor implantation. HT-29, a tumorigenic, non-metastatic human colon carcinoma cell line and A-549, a non-small cell human lung cancer cell line, were obtained from the American Tissue Type Collection (Rockville, Md.). Cells were passaged twice weekly with a 1:2 split and cultured in Dulbecco's modified Eagle's medium (DMEM:F12) supplemented with 10% fetal bovine serum (HyClone, Ft. Collins, Colo.). For inoculation, approximately 10⁶ cells in 0.1 ml of media were injected subcutaneously into the right flank of female severe combined immunodeficient (SCID) mice of ages 5 to 6 weeks (obtained from the Arizona Cancer Center Experimental Mouse Shared Services). Mice developed palpable tumors within a week of inoculation. Tumors were allowed to grow to 100-500 mm³ prior to imaging. All animal protocols were approved by the University of Arizona Institutional Animal Care and Use Committee (IACUC).

Treatments. PX-478 (S-2-amino-3-[4′N,N,-bis(2-chloroethyl)amino]-phenyl propionic acid N-oxide dihydrochloride) was provided by Prolx Pharmaceuticals (Tucson, Ariz.) and was prepared fresh each day in 0.9% NaCl as a 10 mg/ml solution and administered intraperitoneally (i.p.) to the mice within 30 minutes of preparation. Mice were treated with either vehicle or with 125 mg/kg PX-478 and were studied 2, 12, 24, or 48 hours later. A minimum of eight animals were examined with MRI at each time point (4 to 6 controls and 4 to 6 treated). An additional 36 h time point was included in the DW-MRI protocol. For imaging, mice were anesthetized using 1.0-2.0% isoflurane carried in oxygen. Body temperature was maintained at 37° C. with a circulating water blanket and was monitored using a rectal Luxtron fluoroptic thermometer (Luxtron, Santa Clara, Calif.). Contrast agent, Gd-DTPA (Gadolinium-diethylenetriamine-pentaacetic acid) coupled to albumin (Gd-BSA, 0.6 mg/g in 0.15 ml saline), was injected via a tail vein catheter comprising a 30-gauge needle connected to PE-20 polyethylene tubing. The Gd-BSA was synthesized by the Arizona Cancer Center Synthetic Chemistry Core. The human anti-VEGF antibody Avastin™ (Bevacizumab, Genentech, San Francisco, Calif.) was administered intravenously (i.v.) at a dose of 20 μl/30 g.

Magnetic Resonance Imaging. All imaging was performed on a 4.7 T horizontal bore MR imager (Bruker, Billerica, Mass.). Mice were positioned into a 24 mm ID Litzcage coil (Doty Scientific, S.C.). Sagittal scout images were obtained to determine the position of tumors.

DW-MRI methodology. Contiguous axial 2.0 mm slices covering the entire tumor were imaged as per the following protocol. Diffusion-weighted images were obtained using the DIFRAD sequence (48), with the typical acquisition parameters: [TR=2 s, TE=36 ms, Δ=13 ms, δ=5 ms, matrix size=128×28, FOV=4×4 cm, where δ and Δ represent the duration and separation of diffusion gradients, respectively. At each slice location, images were obtained at three b values (25,500, and 950 sec/mm²) [b=γ²G²_{d}∂(Δ−^{∂}/3)]. where G_{d} is the strength of the diffusion weighting gradient and γ is the gyromagnetic ratio for protons. Images were reconstructed using a filtered back projection algorithm of magnitude data, to minimize motion artifacts. ADCw maps were generated by fitting the three b-values to the Stejskal-Tanner equation, S=Sₒe^{-bADCw} where Sₒ is the signal intensity in the absence of diffusion weighting, and S is the signal intensity with diffusion weighting. ADCw maps were analyzed using programs written in Interactive Data Language (Research Systems, Boulder, Colo.). Hand-drawn regions of interest (ROIs) corresponding to tumor localized on the scout scans were cloned onto the ADCw maps, and ADCw distribution histograms were obtained for each tumor. For each time point (2, 12, 24, 36, and 48 h after vehicle or PX-478 injection), two groups (one control and one treated) of 4 to 6 mice were imaged. In addition, 4 mice were monitored over the full time-course, independently of the DCE-MRI protocol in order to confirm the pattern observed on separate groups of mice.

DCE-MRI acquisition and analysis. Contiguous axial 2.0 mm slices covering the entire tumor as well as a slice over the kidneys were imaged in the following protocol. A proton-density-weighted (TR=8 s, TE=5.9 ms, NA=2, FOV=4×4 cm) and a T1 weighted spin-echo image (TR=300 ms, TE=5.9 ms, NA=8, FOV=4×4 cm) were collected prior to injection of contrast. A dynamic series of spin-echo images (TR3=00 ms, TE=5.9 ms, NA=4, F0V=4×4 cm, NR=19) were collected over 45 minutes, with the contrast agent solution being injected during repetitions 2-5.

Signal enhancement in the DCE data was converted to albumin-Gd-DTPA concentration using the relaxivity measured in vitro at 37° C. (1.08 L/g-s) and assuming a linear relationship between Gd concentration and relaxation rate enhancement. This [albumin-Gd-DTPA] vs. time data was fitted to a straight line for each pixel, to obtain a slope (related to vascular permeability) and y-axis intercept (related to the vascular volume).

The vascular volume (VV) parameter measured in tumor pixels was normalized to the mean value obtained in an ROI placed on muscle in the same animal and multiplied by 5% (˜VV fraction of the muscle) to convert it to the vascular volume fraction of the tumor. In order to be able to compare values between different mice, the slope parameter was normalized for Gd dose as follows for each mouse. The mean slope parameter calculated from pixels falling within the vena cava was used to normalize the slope determined in the tumor. The vena cava was identified using a hand-drawn region of interest (ROI) of approximately 5 to 10 pixels. Data analysis was performed using programs written in Interactive Data Language (Research Systems, Boulder, Colo.).

Antitumor studies. The doses of PX-478 used for antitumor studies were 80 mg/kg daily for 5 days for the HT-29 colon cancer xenograft mice and 100 mg/kg daily for 5 days for the A-549 lung cancer xenograft mice. There were 8 mice in each group. Tumor volume was measured twice weekly until the tumor reached 2,000 mm³, or became necrotic, at which point the animals were euthanized. Orthogonal tumor diameters (dₛₕₒᵣₜ and d_{long}) were measured twice weekly with electronic calipers and converted to volume by the formula volume=(dₛₕₒᵣₜ)2×(d_{long})÷2. Log₁₀cell kill was calculated by the formula logio cell kill=(tumor growth delay [day])÷(tumor doubling time [day]×3.32). One-way analysis of variance using the General Linear Model was used to test for the effect of treatment on tumor growth rate and growth delay.

HIF-1α Immunohistochemistry. Paraffin embedded tumor sections were heated at 60° C. for 30 minutes and rehydrated through xylene and graded alcohols. Antigen retrieval was at 40 mm at pH 9.0 for HIF-1α. The slides were blocked for 30 minutes in 4% milk, 1% goat serum, 0.1% thimerosal in phosphate buffered saline (PBS). After blocking, the slides were processed using Endogenous peroxidase activity was quenched using a hydrogen peroxide-based inhibitor (DAB Basic Detection Kit, Ventana Medical Systems, Tucson, Ariz.) and endogenous biotin was blocked using an AB Blocking Kit (Ventana Medical Systems). The slides were incubated for 32 minutes at 42° C. with the mouse monoclonal anti-human HIF-1α (Transduction Labs, Lexington, Ky.) at 10 μg/ml. A biotinylated universal secondary antibody which recognized mouse IgG/IgM was applied, followed by horse radish peroxidase-conjugated avidin, DAB/hydrogen peroxide and a copper enhancer. The slides were dehydrated through graded alcohols, toluene, and xylene and cover slipped using Vectamount (Vector Laboratories, Burlingame, Calif.). HIF-1α staining was normalized to the staining of an on-slide control of hypoxic HT-29 colon cancer cells.

VEGF detection. Plasma was collected into EDTA tubes and tumors were removed and immediately snap frozen in liquid nitrogen. Tumors were then placed in buffer (10 mM Tris/HCl pH 7.4, and 100 mM NaCl) and homogenized using a PowerGen 125, Fisher Scientific, Pittsburgh, Pa. The suspension was then centrifuged twice at 8,000×g at 4° C. for 15 min. Protein was quantitated in supernatant using the Pierce BCA assay. VEGF levels were quantitated in plasma and tumor lysates using both human (hVEGF) and mouse VEGF (hVEGF) ELISAs (R&D systems, Minneapolis, Minn.), according the manufactures' instructions.

Statistical analysis. Data are presented as the mean and 95% confidence intervals (CIs). Two-tailed Student's t-tests, ANOVA, or Mann-Whitney Rank Sum tests were used where appropriate. A P value <0.05 was considered to be statistically significant.

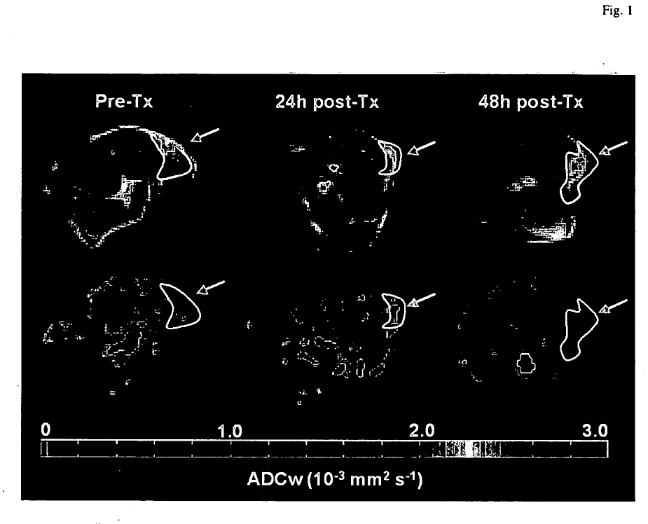

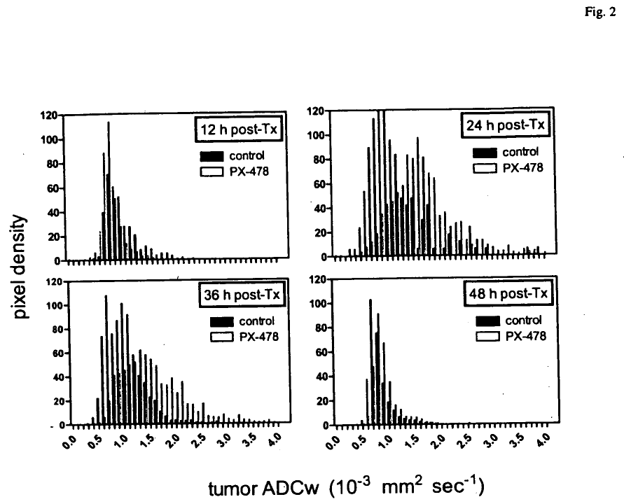

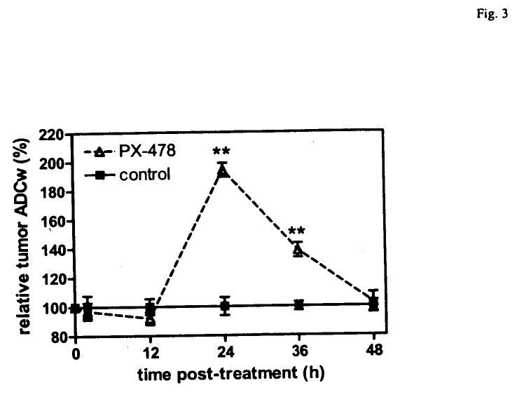

This example illustrates the effect of PX-478 on HT-29 tumor ADCw.DW-MRI was used to detect the early response of HT-29 tumor xenografts to the antitumor agent PX-478. A single gradient direction was used because previous studies have shown the absence of anisotropy in extracranial tumor models. Time-course ADC maps from a representative animal are shown in FIG. 1. Regions of interest (ROIs) defining the tumor were used to generate histograms of tumor ADCw values. ADCw histograms of individual tumors were then summed for each time point (FIG. 2). A right shift in tumor water diffusion beginning by 24 h after therapy is shown in FIG. 2. Water diffusibility was still increased by 36 h post-treatment and appeared to return to pre-treatment values by the second day after therapy. Changes in mean tumor ADCw values over with time post-treatment are also presented in FIG. 2. No change in ADC distribution was observed in sham-treated animals (FIG. 2). At early time points (2 and 12 h), ADCw values were not significantly different between control and treated groups. A substantial increase in mean relative tumor ADCw was observed for the treated groups at 24 and 36 hours post-treatment (+94.5%, 95% C182.5 to 106%, P0.005 and +38.4%, 95% C126.3 to 50.5%, P0.01, respectively) (FIG. 3), before returning to pre-treatment mean ADCw values by 48 h post-treatment (non significant change of +2.5%, 95% C1 −3.2 to +8.2%, P=0.38). This significant change in ADCw (by 24 h) occurs sooner than in other reports.

### EXAMPLE 2

This example illustrates the effects of PX-478 on HT-29 tumor DCE-MRI parameters. Extravasation of the Gd-BSA was assumed to be describable by a permeability-limited two-compartment model with unidirectional transport of contrast agent on the timescale of the DCE-MRI experiments.

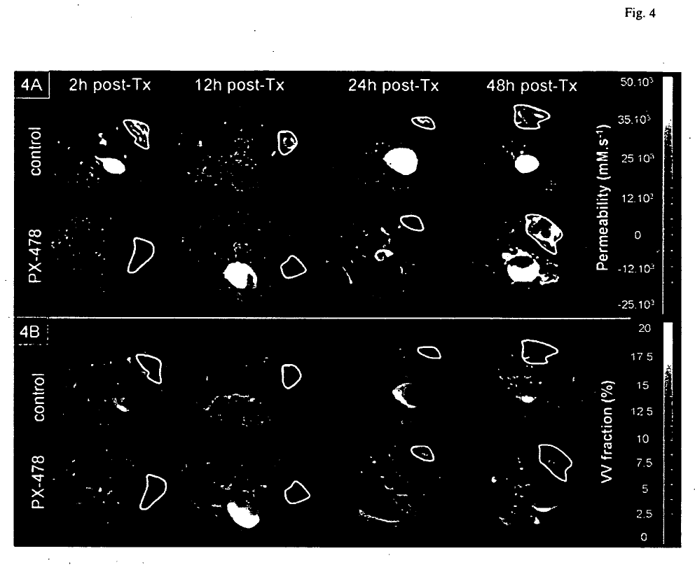

Parameter maps of permeability and vascular volume fraction were created to visualize the heterogeneity of tumor hemodynamic parameters. Heterogeneities in the distributions of pharmacokinetic parameters have previously been shown in experimental as well as in human tumors. Typical permeability (P) and vascular volume fraction (VV) maps at each time point are shown in FIG. 4. Tumors were identified on proton density-weighted images and delineated by hand-drawn ROIs. Tumor vascular permeability is dramatically decreased in the PX-478 group 2, 12, and 24 h after treatment in comparison with the control group (FIG. 4A). This decrease is no longer observed by 48 h after treatment. Although some individual changes (positive or negative) in tumor vascular volume fraction were sometimes observed (see FIG. 4B, 2 and 24 post-Tx), the mean change between groups was not statistically significant.

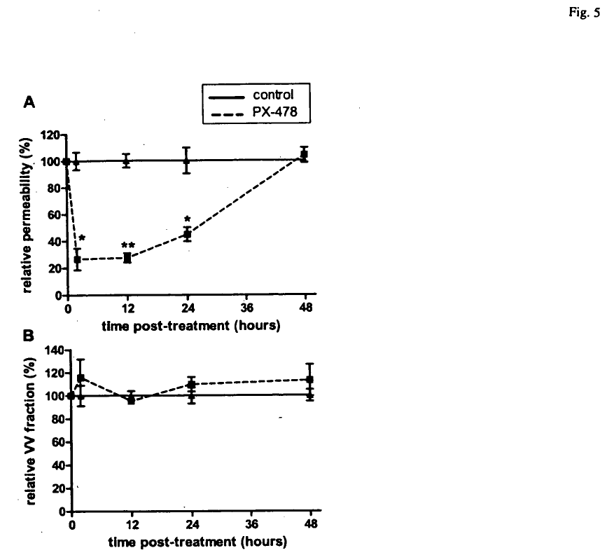

Time courses of mean normalized permeability values and of mean VV fraction values are presented in FIG. 5 (relative data) and Table 1 (absolute values). A rapid decrease in tumor blood vessel permeability was observed within 2 hours after drug administration compared to control tumors, with a mean reduction of 73.3% (95% CI 38.3 to 108.3, P=0.012). The decrease in permeability was still about 72.4% about 12 hours after treatment (95% CI 54.7 to 90.1%, P=0.003). The effect progressively decreased in the later time points, with a mean reduction of 55.0% (95% CI 29.7 to 80.3%, P=0.02) at 24 hours post-treatment and a return to control values at 48 hours (+3.9%, 95% CI −24.2 to +32.0%, P=0.71, not significant). By contrast, the vascular volume fraction of the tumor was not significantly modified at any time point and remained unchanged between control and treated tumors.

TABLE 1
Normalized values of DCE-MRI enhancement parameters
after treatment with PX-478 or Avastin

| | | 1 h post-Tx | | 2 h post-Tx | | 12 h post-Tx | | 24 h post-Tx | | 48 h post-Tx | |
|---|---|---|---|---|---|---|---|---|---|---|---|
| Tumor | | | VVf | | VVf | | VVf | | VVf | | VVf |
| model | Tx | nP | (%) | nP | (%) | nP | (%) | nP | (%) | nP | (%) |
| HT-29 | Control | 0.65 | 6.4 | 0.62 | 6.5 | 0.63 | 6.0 | 0.62 | 6.0 | 0.60 | 5.8 |
| | | [0.55; | [4.7; | [0.40; | [2.8; | [0.42; | [4.5; | [0.24; | [3.3; | [0.56; | [3.0; |
| | | 0.76] | 8.0] | 0.84] | 10.2] | 0.83] | 7.5] | 1.00] | 8.6] | 0.63] | 8.7] |
| | PX-478 | n.d. | | 0.17* | 7.5 | 0.17** | 5.7 | 0.28* | 6.6 | 0.62 | 6.6 |
| | | | | [−0.11; | [1.6; | [0.04; | [4.9; | [0.07; | [4.2; | [.46; | [4.3; |
| | | | | 0.44] | 13.4] | 0.31] | 6.5] | 0.48] | 8..9] | 0.78] | 9.0] |
| | Avastin | 0.16** | 4.4 | | | | n.d. | | | | |
| | | [0.08; | [3.8; | | | | | | | | |
| | | 0.25] | 4.9] | | | | | | | | |
| A-549 | Control | n.d. | | 0.35 | 6.3 | | | n.d. | | | |
| | | | | [0.32; | [5.1; | | | | | | |
| | | | | 0.38] | 11.4] | | | | | | |
| | PX-478 | | | 0.34 | 6.0 | | | | | | |
| | | | | [0.31; | [3.7; | | | | | | |
| | | | | 0.37] | 9.7] | | | | | | |

Normalized permeability (nP) and vascular volume fraction (VVf) values presented as means and 95% CI [min; max] for control (carrier injection), PX-478 (125 mg/kg i.p.), and Avastin (20 μl/30 g i.v.) groups.
*p < 0.05,
**p < 0.01 relative to the control group (two-tailed t-tests). Note that the permeability is significantly decreased 2, 12, and 24 h after treatment with PX-478 and within 1 hour after treatment with the anti-VEGF antibody Avastin and that the VVf is only affected by Avastin.

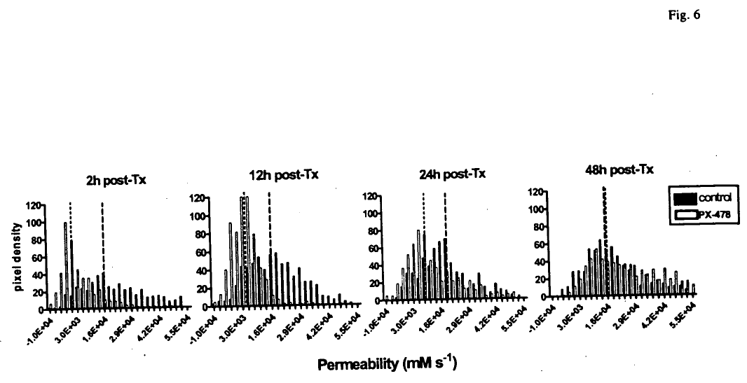

Histogram analyses of these data lose spatial information yet retain the distribution of values for quantitative analyses. FIG. 6 shows histogram data summed for all animals in each group. Control tumors at each time point (filled bars in each plot) were characterized by heterogeneous and broad distributions of permeability values at all time points. In contrast, treated tumors showed more homogeneous and narrow histograms centered around much lower values at 2, 12, and 24 h (open bars). The range of median of the distribution of permeability values returned to control levels at 48 hours. These data can also be further reduced to median values (dashed vertical lines in each population), which were significantly decreased in the treated groups 2, 12, and 24 h after treatment.

### EXAMPLE 3

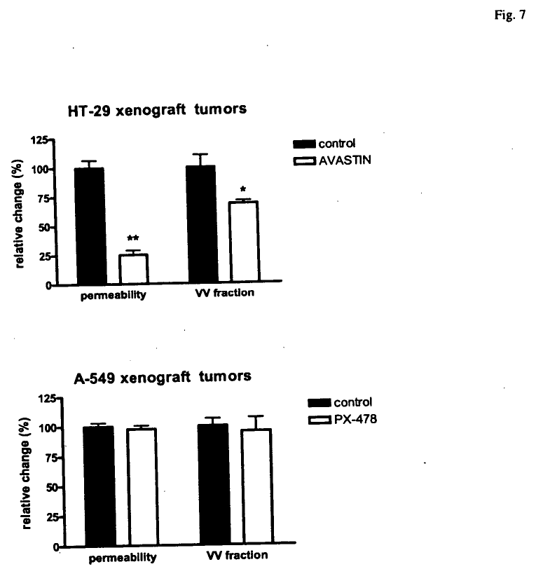

This example illustrates the effects of anti-VEGF antibodies on HT-29 tumor DCE parameters. In order to assess the ability of the MMCM DCE technique to detect acute changes after treatment with an antitumor agent aimed at decreasing VEGF in this tumor model, human anti-VEGF antibody bevacizumab (Avastin™) was administered to HT-29 tumor bearing mice. A 75% decrease in vascular permeability was observed within an hour of injection of the antibody (95% CI 60.2 to 89.8%, P<0.0001), similar to the changes observed 2 h and 12 h after PX-478 administration (FIG. 7A, Table 1). The anti-VEGF antibody treatment also induced a significant 31.5% (95% CI 25.3 to 37.7%, P=0.023) decrease in vascular volume fraction, unlike treatment with PX-478 (FIG. 7A, Table 1).

### EXAMPLE 4

This example illustrates the effects of PX-478 on A-549 tumor DCE parameters. A-549 non-small cell lung tumors are resistant to PX-478 and were therefore used as negative controls for the DCE-MRI protocol. No significant change was observed for either tumor permeability or vascular volume fraction (FIG. 7B, Table 1). These data suggest that the changes observed on HT-29 xenografts after administration of PX-478 are connected to the sensitivity of this tumor model to the drug. Notably, the untreated permeability values of A-549 tumors were lower than the control values obtained in HT-29 tumors, suggesting that base line permeability may be prognostic for the anti-tumor effects of PX-478, although further investigation is required.

### EXAMPLE 5

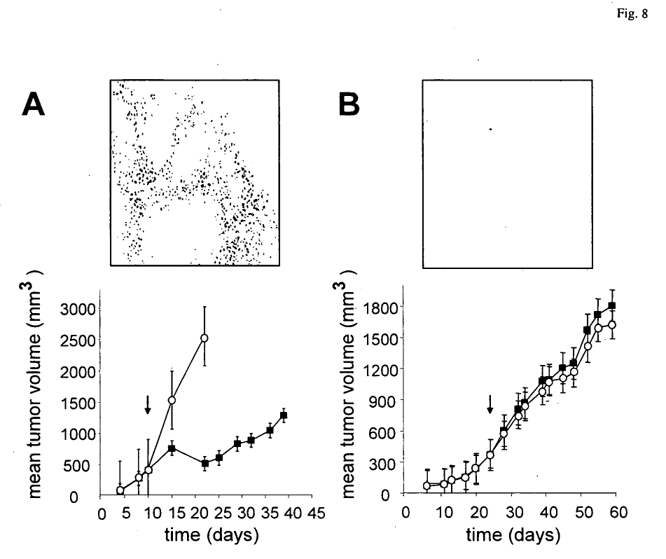

This example illustrates the antitumor effect of PX-478 on HT-29 and A-549 xenografts, HIF-1α staining and VEGF detection. HT-29 colon cancer xenografts exhibited staining for HIF-1α while A-549 non small cell lung cancer xenografts showed very little staining (FIG. 8). The A-549 lung cancer xenografts showed no growth inhibition when treated with PX-478 100 mg/kg ip daily for 5 days whereas the HT-29 colon cancer xenografts exhibited a tumor growth delay of 16 days with a calculated log cell kill of 1.6 (P<0.05). The lack of responsiveness to PX-478 by A-549 tumors may be due to the lack of HIF-1α expression in these tumors compared to HT-29 xenografts (FIG. 8). The lower permeability observed may be explained by the lower expression of VEGFA, a HIF-1 target gene. Levels of VFGFA are also markedly lowered in A549 tumors vs HT-29 tumors (50.12 pg/μg, vs 1.81 pg/μg, 95% CI 1.23 to 2.40 pg/μg, P=0.012, Mann-Whitney Rank sum test) as measured by ELISA.

The activity of PX-478, an inhibitor of HIF-1α in experimental tumors was evaluated on HTF-29 human colon xenografts using both dynamic contrast enhanced and diffusion weighted MRI. PX-478 induced a substantial reduction in tumor blood vessel permeability as early as two hours after a single dose of 125 mg/kg, which persisted until 24 h post-treatment, and had returned to control values by 48 hours. The tumor vascular volume fraction was not significantly altered over the same time course. Although the time course of response was different for diffusion MRI, tumor ADCw was also shown to be an early marker of tumor response. No change in tumor ADCw could be observed at very early time points, but a significant increase was shown 24 and 36 hours after treatment, having returned to control values by 48 h post-treatment.

Tumor permeability to MMCM has been used in the preclinical setting in order to assess the efficacy of different antiangogenic therapies (27,30,32,34,50). MMCM-enhanced MRI has been demonstrated to be capable of monitoring the direct antivascular effects of anti-VEGF antibody treatment in xenografts (51-53). A decrease in tumor vascular parameters (K^{tans}) in animal human tumor xenograft models following treatment with the small molecule VEGF-receptor tyrosine kinase inhibitors ZD6474 (54) and PTK787/ZK222584 (55,56), and anti-VEGF antibody (57-59) has been measured by DCE-MRI using clinically approved small molecule contrast agents. DCE-MRI studies in patients with colon cancer receiving PTK 787/ZK222584 as part of Phase I trials, while showing heterogeneity in tumor vascular permeability response, have shown a significant correlation between tumor permeability and vascularity and the dose of PTK 787/ZK222584, with patients with stable disease having a significantly greater reduction in permeability (K^{trans}) (55). Patients receiving anti-VEGF antibody as part of Phase 1 trial have also exhibited a reduction in tumor K^{trans} measured by DCE-MRI after the first treatment (60).

Acute changes within an hour following anti-VEGF antibody therapy using the large molecular contrast agent, GdBSA were observed. This suggests that the reduction in vascular permeability parameters measured by DCE-MRI is related to changes in tumor VEGF levels. In this context, PX-478 has been shown to decrease both HIF-1α and VEGF staining in HT-29 tumors. However, the time course for the decrease in HIF-1α and VEGF was different from the changes in permeability measured by DCE-MRI. Previous studies showed that both HIF-1α and VEGF decreased within two hours, yet the levels had returned to control values by 8 hours after treatment. In contrast, in the methods of the present invention the vascular permeability estimated from MMCM kinetics was still reduced 24 hours after treatment.

In patients, increased VEGF expression has been correlated with the progression of colon carcinoma and with the development of colon cancer metastasis. In node negative primary colon cancer, elevated tumor VEGF has been correlated with decreased patient survival. Also, increased tumor VEGF expression has been associated with increased tumor angiogenesis and metastasis of human gastric cancer. However, the estimation of VEGF levels is now more controversial as an accurate marker of therapeutic efficacy. Clinical studies focused on the relationship between angiogenic markers (microvascular density or VEGF levels) and quantitative DCE-MRI enhancement data and have shown mixed results. Su et al. concluded that the lack of correlation could be partly due to the inability of DCE-MRI with low molecular weight agents to reveal the true vascular function within the tumor. Bhujwalla et al. recently described the antiangiongenic effect of the fumagillin derivative TNP-470 by MMCM DCE-MRI. They observed a heterogeneous response, with some regions of decreased permeability and some regions with increased permeability values, resulting in an apparent lack of overall response based on the average value of tumor permeability, while ELISA assays detected an increase of tumor VEGF. DCE-MRI was shown to be a more reliable marker by taking into account the tumor heterogeneity. The methods provided herein suggest that DCE-MRI using MMCM might be a more sensitive measure of functional tumor permeability or that permeability factors other than VEGF might be involved in the response to PX-478.

Importantly, a lack of change in permeability in A-549 tumors between control and treated tumors was observed. This correlates well with the inability of PX-478 to induce growth delays in A-549 tumors. The base line permeability values were lower in A549 than in HT-29 tumors.

If it has been suggested in the past that DCE-MRI could be used to monitor clinical response to anti-VEGF and inhibition of angiogenesis, the data disclosed herein suggest that DCE-MRI may also be useful to assess the response to inhibition of HIF-1. A tumor with low HIF-1α staining which was not responsive to anti-HIF-1 therapy also had a very low vascular permeability measured by DCE-MRI, suggesting that DCE-MRI may also be useful clinically for screening and preselecting patients for therapy with anti-HIF-1 and other anti-angiogenic therapies. Examples of anti-HIF-1 compounds include, but are not limited to, PX-478, geldanamycin, inhibitors of Topoisomerase I, anti-HIF-1 antibodies, etc. Examples of anti-angiogenic compounds that may be used in therapeutic regimens include, but are not limited to, compounds such as angiostatin, endostatin, fumagillin, non-glucocorticoid steroids and heparin or heparin fragments and antibodies to one or more angiogenic peptides such as αFGF, βFGF, VEGF, IL-8 and GM-CSF.

Diffusion weighted MRI is able to detect early changes in the morphology and physiology of tissues after antineoplastic therapies. An increase in tumor ADCw is thought to be the result of changes in either cell membrane permeability, or cell shrinkage. Both of these are associated with cell death and result in the modification of the intracellular to extracellular water populations ratio. Parameters such as cell density and necrotic fraction have indeed been monitored with diffusion MRI. The methods of the present invention detect an increase in tumor ADCw that is consistent with other studies using other tumors and drugs. The methods disclosed herein detect an increase in ADCw that is correlated with the ultimate tumor response, whether by apoptosis or other means of cell death. Notably, the current data resulting from the methods disclosed document the earliest significant increase in chemotherapy-induced ADCw. Previous reports have indicated that the earliest significance was not reached until 48 h following therapy.

The combination of dynamic and diffusion weighted MRI in the follow up of chemotherapy has been used in the past and has been proven to be of good predictive value for therapy outcome in patients with primary rectal carcinoma. Embodiments provided herein wherein the acquisition of both diffusion-weighted and dynamic contrast enhanced images are utilized in a single protocol on the same animal allowed the data to be co-registered and to compare the two techniques. Therefore, the dynamic range (DR) can be defined as the maximum change relative to the variance of controls. For these studies the DR was higher for DW-MRI (maximum effect at 24 h, DR=8.7) than for DCE-MRI (maximum effect at 2 h, DR=3.2). Tumor ADCw was thereby shown to be a sensitive and early marker of tumor response. Nonetheless, the DCE-MRI response preceded the diffusion response and opens up the possibility of monitoring acute effects of drugs in vivo. The combination of the two techniques gives unique insights into the complex response of HT-29 tumors to PX-478 by showing very early changes in vascular permeability followed by large changes in cellularity. Considering the magnitude of response of HT-29 xenografts to PX-478 observed with early and sensitive markers, the non-invasive monitoring of PX-478 by DCE and/or diffusion MRI may be of particular interest in the clinic.

### EXAMPLE 6

This example illustrates the response of the tumor to PX-12 treatment, as detected using DCE-MRI. Extravasation of the Gd-BSA was assumed to be describable by a permeability-limited two-compartment model with unidirectional transport of contrast agent on the timescale of our DCE-MRI experiments. Thus, the coefficient of endothelial permeability and the fractional plasma volume could be estimated from straight line fits of the concentration vs. time data for each pixel. These parameters were also averaged in regions of interest covering the whole tumor, in all slices.

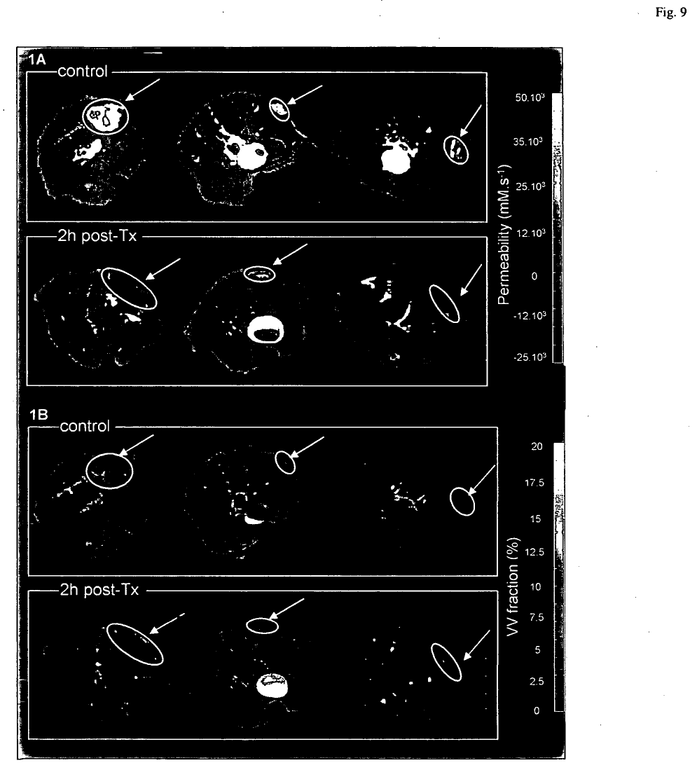

Parameter maps of permeability and vascular volume fraction were created to visualize the heterogeneity of tumor hemodynamic parameters. These maps provided unique insights into the complex response of tumors to PX-12. Heterogeneities in the distributions of pharmacokinetic parameters have previously been shown in experimental as well as in human tumors. Typical examples of permeability maps of control and treated tumors 2 h after vehicle or carrier injection are shown in FIG. 9A. Note that the tumor blood vessel permeability was considerably lower 2 h after treatment with drug, compared to control tumors. There was no significant change in the average tumor VV fraction induced by the drug (FIG. 9B).

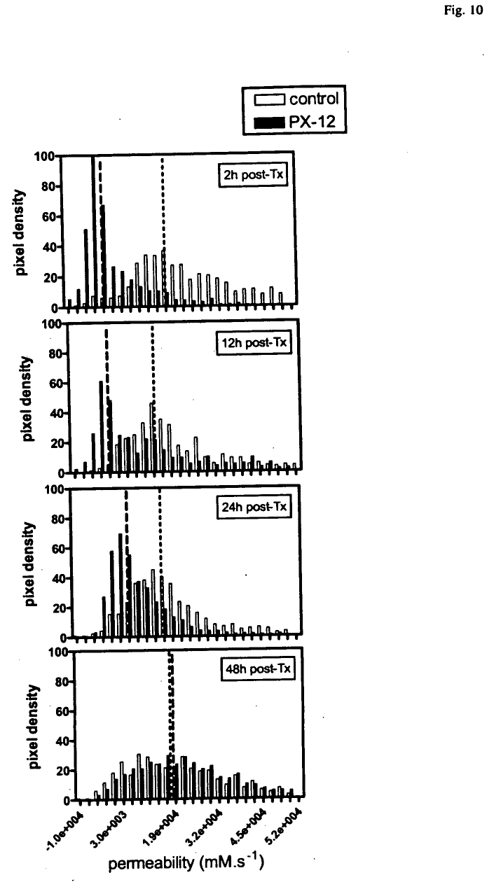

Histogram analyses of these data lose spatial information yet retain the distribution of values for quantitative analyses. FIG. 10 shows histogram data summed for all animals in each group. Control tumors at each time point (open bars in each plot) were characterized by heterogeneous and broad distributions of permeability values, and this was invariant between time points. In contrast, treated tumors showed homogeneous and narrow histograms centered around much lower values at 2, 12, and 24 hr (FIG. 10). Note that the distribution of permeability values returned to control levels within 48 hours. These data can also be further reduced to median values (dashed vertical lines in each population), which were significantly decreased in the treated groups 2 hr, 12 hr, and 24 hr after treatment.

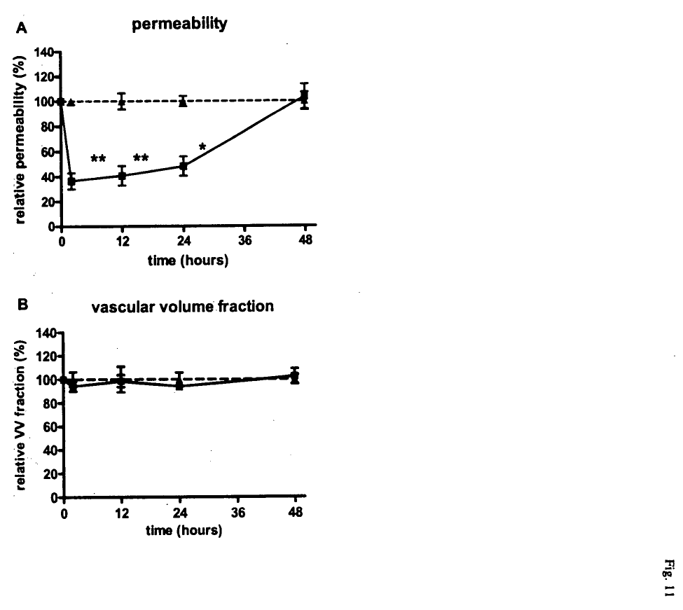

The time course presented on FIG. 11 shows a rapid decrease in tumor blood vessel permeability within 2 hours after PX-12 injection in comparison with untreated tumors (FIG. 3A). This substantial reduction was evident across all tumors studied, with a mean reduction of 63.4% (±11.3%, P<0.01). The decrease in permeability was still considerable 12 hours after treatment, with a mean reduction of 59.2% (±11.2%, P<0.01). The effect progressively decreased in the later time points, with a mean reduction of 51.6% (±7.2%, P<0.05) at 24 h post-treatment and a return to control values at 48 hours (+3.4±17.6%, not significant). By contrast, the vascular volume fraction of the tumor was not significantly modified at any time point and remained comparable between control and treated tumors as shown in FIG. 11B.

### EXAMPLE 7

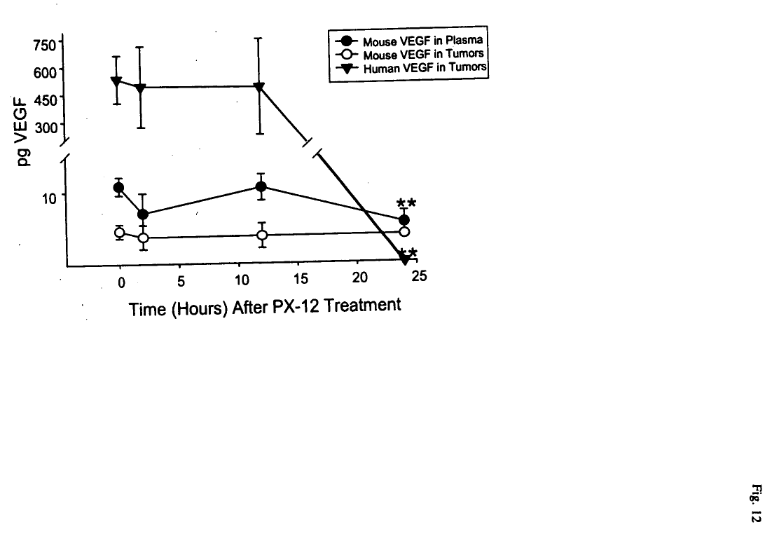

This example illustrates the effects of PX-12 on plasma and tumor VEGF levels, as measured by ELISA assays. In these xenografts, hVEGF is derived from the tumor and mVEGF from the host vasculature and stromal tissue, resulting in considerably higher hVEGF than mVEGF in the tumor. Although the endothelium is mouse derived, hVEGF inhibition in xenograft models has been reported to cause obliteration of the host tumor vasculature, implying that hVEGF is active at mouse VEGF receptors VEGFRI (Flt-i) and VEGFR2 (KDR). Therefore, levels of both mouse and human VEGF were measured in the tumor and plasma samples collected. A significant decrease in hVEGF levels was found within the tumors after 24 hours of treatment, but not at early time points (FIG. 12). Mouse VEGF showed only a small decrease starting at 2 h but this decrease did not reach statistical significance at any of the time points measured.

Some tumors are reported to secrete VEGF into the plasma which can then act on endothelial cells in a paracrine manner or on tumor cells in an autocrine loop if the tumor expresses the VEGF receptors VEGFRI or VEGFR2. Plasma hVEGF expression was too low to be detected in the plasma from the HT-29 xenografts. However, PX-12 has been observed to cause a decrease in circulating levels of VEGF in patients treated with the agent as soon as 4 hours post drug administration. In this example the mouse plasma VEGF levels were detectable and showed a decrease at 2 hours post treatment and this decline reached statistical significance (P<0.02) at 24 h post PX-12.

PX-12 is an investigational cancer drug that inhibits Trx-1 signaling. It has been shown to decrease HIF-1α protein levels, the expression of downstream target genes such as VEGF, and the microvessel density in different tumor models, including HT-29 human colon carcinoma xenografts. A recent phase I study in patients with advanced malignancies revealed antitumor activity. Additionally, patients in this study showed a decrease in plasma VEGF levels. Embodiments of the present invention utilize dynamic contrast-enhanced MRI with MMCM to assess hemodynamic changes in FIT-29 tumor xenografts after treatment with PX-12. The slope of the time-dependent enhancement produced by the MMCM Gd-BSA was used as a marker of vascular permeability, and was measured at 2, 12, 24, and 48 h after drug or vehicle injection. PX-12 was shown to cause a significant reduction in tumor vascular permeability within 2 hours of administration, with significant reduction apparent at 24 hours post-treatment, returning to pre-treatment values by 48 h after treatment. The y-intercept of the time-dependent enhancement produced by Gd-BSA was used as a marker of vascular volume fraction, and this was not affected by PX-12 at any of the time-points measured.

Macromolecular DCE-MRI has been used to follow changes in vascular volume and permeability induced by anti-angiogenic therapies in a preclinical setting. It has also been demonstrated that MMCM-enhanced MRI is capable of monitoring the anti-vascular effects of anti-VEGF antibody treatment in xenografts. In that study, large reductions in permeability were seen within 24 h of a 3 day treatment that were not accompanied by a change in fractional plasma volume. It was later confirmed that both intermediate and large molecular contrast agents were suited to monitor tumor response to VEGF antibodies in experimental tumors where significant reductions in permeability as well as in fractional plasma volume were observed. The inventors have previously shown that acute changes in both permeability and vascular volume parameters in HT-29 xenogfraft tumors were observed within an hour of a single dose anti-VEGF antibody treatment.

Methods utilized herein reveal that PX-12 produces a notable reduction in tumor vascular permeability, however no changes in tumor vascular volume fraction were observed. Other investigators have also observed this pattern of response following administration of anti-angiogenic treatments, such as the kinase inhibitor PTK787/KZ222854 in an experimental breast cancer model. The lack of change in fractional plasma volume was not a function of the current tumor model, since SU6668 in this same system caused a decrease in DCE-MRI measured vessel permeability as well as in fractional plasma volume by 24 h post-treatment.

Tumor and plasma VEGF (human and mouse) levels were monitored in order to test the hypothesis that changes in MRI-measured hemodynamic parameters may be correlated to this centrally important angiogenic factor. Since angiogenesis is essential for the growth, invasion, and metastasis of cancers, the stimulatory factors may also be used as prognostic factors. Nevertheless, clinical studies focused on the relation between angiogenic markers (microvascular density or VEGF levels) and quantitative DCE-MRI enhancement data have shown mixed results. Su et al. concluded that the lack of correlation could be partly due to the inability of DCE MRI with low molecular weight agents to reveal the true vascular function within the tumor. Bhujwalla et al. recently described the anti-angiogenic effect of the fumagillin derivative TNP-470 by MMCM DCE-MRI. They observed a heterogeneous response, with some regions of decreased permeability and some regions with increased permeability values, resulting in an apparent lack of overall response based on the average value of tumor permeability, while ELISA assays detected an increase of tumor VEGF. DCE-MRI was shown to be a more reliable marker by taking into account the tumor heterogeneity.

The examples described herein revealed notable decreases in tumor VEGF levels 24 h after treatment, similar to what was observed with DCE-MRI. However, a lack of correlation was seen at earlier time points, since no significant change in tumor VEGF levels could be observed 2 or 12 hours post-treatment. This data might suggest that other permeability factors besides VEGF may be affected by PX-12 or that DCE-MRI is a more sensitive measure of functional tumor VEGF than ELISA. Human VEGF measured in the tumor by ELISA assay seems to be the most relevant factor, since the xenografted tumor cells are of human origin. It is however possible that the neovasculature in the tumors depend on VEGF secreted by both the tumor and host tissues. A further caveat to these data is the fact that the ELISA assay is based on monoclonal antibodies that recognize only the VEGF-A-165 splice variant isoform of this hormone. Hence, it is possible that other forms of VEGF are related to these hemodynamic changes.

Few studies considered very early time-points following anti-angiogenic treatment. Beauregard et al. studied the effect of two anti-vascular agents, the tubulin binding combretastatin A4 phosphate and the TNF activator DMXAA, on HT-29 xenografts up to three hours post-treatment. Interestingly, they observed different patterns of response with the two drugs: CA4P showed only a small decrease in tumor perfusion, while DMXAA considerably decreased perfusion. DCE-MRI also allowed us to detect an early response of HT-29 tumors to PX-12 treatment. Although widely used in (pre)clinical applications, both microvessel density and VEGF levels have been controversial as being good indicators of the therapeutic efficacy of anticancer drugs. The possible disconnect between VEGF and DCE-MRI results may indicate that imaging provides a more reliable marker of tumor response to this drug, although this remains to be shown. For example, the relevance of the permeability decrease to clinical response has yet to be established. Nonetheless, DCE-MRI time course studies in experimental models may be helpful in the design of clinical trials and imaging endpoints.

Although the present invention has been described in considerable detail with reference to certain preferred embodiments thereof, other versions are possible. Therefore the spirit and scope of the appended claims should not be limited to the description and the preferred versions contained within this specification.

What is claimed is:

1. A method for screening and preselecting patients for anti-VEGF therapy, anti-HIF-1 therapy or anti-thioredoxin therapy comprising:
administering a macromolecular contrast medium to said patient;
imaging the change in signal intensity of diffusion weighted and spin-echo weighted images over time in a tumor to obtain a signal intensity; and
determining tumor vascular structure and permeability.

2. The method of claim 1, wherein said therapy is administration of a compound selected from the group consisting of VEGF inhibitors, thioredoxin inhibitors and HIF inhibitors.

3. The method of claim 1, wherein said anti-HIF-1 therapy is administration of a HIF-1α inhibitor.

4. The method of claim 3 wherein said HIF-1α inhibitor is S-1-amino-3-[4′N,N,-bis(2-chloroethyl)amino]-phenyl propionic acid N-oxide dihydrochloride.

5. The method of claim 1, wherein said anti-thioredoxin therapy is administration of a thioredoxin inhibitor.

6. The method of claim 5, wherein said thioredoxin inhibitor is 1-methylpropyl 2-imidazolyl disulfide.

7. The method of claim 1, wherein said anti-VEGF therapy is administration of bevacizumab.

8. The method of claim 1, wherein said step of determining tumor vascular structure comprises analyzing tumor image maps.

9. The method of claim 1, wherein said step of determining tumor vascular structure comprises analyzing tumor permeability, tissue blood volume, tumor vascularity and capillary permeability.

10. The method of claim 1, wherein said macromolecular contrast medium is selected from the group consisting of superparamagnetic iron oxide particles, nitroxides and paramagnetic metal chelates.

11. The method of claim 1, wherein said macromolecular contrast medium is gallidium.

12. The method of claim 1 further comprising administering said anti-VEGF therapy, said anti-HIF-1 therapy or said anti-thioredoxin therapy following determination of tumor vascular structure.

13. The method of claim 12, wherein said therapy is selected from the group consisting of VEGF antibodies, thioredoxin inhibitors and HIF inhibitors.

14. The method of claim 12, wherein said anti-HIF-1 therapy is S-1-amino-3-[4′N,N,-bis(2-chloroethyl)amino]-phenyl propionic acid N-oxide dihydrochloride.

15. The method of claim 12, wherein said anti-thioredoxin therapy is 1 methylpropyl 2-imidazolyl disulfide.

16. The method of claim 12, wherein said anti-VEGF therapy is bevacizumab.
